(19) United States
(12) Patent Application Publication
(10) Pub. No.: US 2025/0017769 A1
(43) Pub. Date: Jan. 16, 2025

(54) POLYMER-BASED GEL IMPLANT FOR RETINAL THERAPY AND METHODS OF MAKING AND USING THE SAME

(71) Applicant: University of Pittsburgh - Of the Commonwealth System of Higher Education, Pittsburgh, PA (US)

(72) Inventors: Morgan DiLeo, Mars, PA (US); Leah Byrne, Pittsburgh, PA (US); José-Alain Sahel, Pittsburgh, PA (US)

(73) Assignee: University of Pittsburgh - Of the Commonwealth System of Higher Education, Pittsburgh, PA (US)

(21) Appl. No.: 18/710,080

(22) PCT Filed: Nov. 15, 2022
(86) PCT No.: PCT/US2022/049965

§ 371 Date: May 14, 2024

Related U.S. Application Data
(60) Provisional application No. 63/279,908, filed on Nov. 16, 2021.

(51) Int. Cl. A61F 9/00; A61L 27/18; A61L 27/52; A61L 27/54

(52) U.S. Cl. CPC A61F 9/0017; A61L 27/18; A61L 27/52; A61L 27/54; A61L 2400/06

(57) ABSTRACT

Disclosed herein are embodiments of a polymer-based gel implant and methods of making and using the same. The polymer-based gel implant comprises a polymer component and a therapeutic agent. In some embodiments, the polymer-based gel implant can be used to treat and/or prevent retinal diseases and/or retinopathies. The polymer-based gel implant exhibits physical properties that provide the ability to safely place the polymer-based gel implant in an ocular region without undesired diffusion and also to allow for controlled and timely release of the therapeutic agent to a desired region of the ocular region, such as the retina. In particular disclosed embodiments, the polymer-based gel implant can be used for safe and effective gene therapy.

### CROSS REFERENCE TO RELATED APPLICATION

This application claims the benefit of and priority to the earlier filing date of U.S. Provisional Patent Application No. 63/279,908, filed on Nov. 16, 2021, the entirety of which is incorporated herein by reference.

### FIELD

Disclosed herein are embodiments of a polymer-based gel implant for retinal therapy and methods of making and using the same.

### BACKGROUND

The retina is the light-sensitive layer of tissue that lines the inside of the eye and communicates with the brain through the optic nerve. Several diseases involving the retina have been discovered and require treatment. Additionally, retinal degenerations/retinopathies are common and thus treatments preventing and/or reducing the extent of such degenerations/retinopathies are needed. Retinal gene therapy typically is administered by one of two routes: subretinal or intravitreal. Subretinal injections have the advantage of lower doses leading to lower immunogenicity and a high rate of infection due to the localization near the target cells; however, the area that can be accessed is extremely limited and this technique is quite invasive. There can be damage to the fovea and potential reflux into the vitreous cavity, leading to lack of control over the administered dose. Intravitreal injection, while noninvasive and capable of accessing a large area, requires a high dose that can lead to high immunogenicity. A lower % of cells are targeted through this route and reflux out of the eye is common. As such, there is a need in the art for more effective treatments for retinal disease and/or retinopathies and means for administering such treatments.

### SUMMARY

Disclosed herein are embodiments of a polymer-based gel implant, comprising: a polymer component comprising one or more polymer species units, wherein the polymer component is capable of absorbing water such that the polymer component transitions from a gel phase to a liquid phase as a concentration of the polymer component in the polymer-based gel implant decreases; and a therapeutic agent suspended in the polymer component; wherein the polymer-based gel implant is a gel at ambient temperature and comprises water.

Also disclosed herein are embodiments of a method, comprising: providing a polymer-based gel implant of the present disclosure; and implanting the polymer-based gel implant into an ocular region of a subject.

Also disclosed herein are embodiments of a method, comprising treating a retinal disease and/or a retinopathy by implanting a polymer-based gel implant according to the present disclosure in an ocular region of a subject having, or capable of developing, the retinal disease and/or retinopathy.

Also disclosed herein are embodiments of a method of making a polymer-based gel implant according the present disclosure, comprising: hydrating the polymer component by combining it with water to provide a hydrated polymer component; and combining the hydrated polymer component and the therapeutic agent to provide the polymer-based gel implant.

The foregoing and other objects and features of the present disclosure will become more apparent from the following detailed description, which proceeds with reference to the accompanying figures.

### BRIEF DESCRIPTION OF THE DRAWINGS

FIG. 1 is a phase diagram showing the different phases in which a representative polymer-based gel implant can exist as a function of temperature and polymer concentration (wt %) and includes a summary of the progression of the different phases in which the polymer-based gel implant can exist at different time periods.

FIG. 2 is a schematic illustration of a polymer-based gel implant comprising a backing layer material and illustrating how the backing layer material can be used to promote unidirectional delivery of the therapeutic agent to the retina and prevent delivery to other regions of the eye.

FIGS. 3A and 3B are photographic images of a polymer-based gel implant modified with a backing layer material, wherein FIG. 3A shows a normal view of the gel implant wherein the polymer component comprises fluorescein isothiocyanate (FITC) and FIG. 3B shows a fluorescent image of the gel implant, illustrating how the backing layer material prevents FITC dissolution through the backing layer and instead promotes dissolution through one direction of the gel implant.

FIG. 4 is a proton nuclear magnetic resonance spectrum of a representative polymer component, namely octadecane-poly(ethylene glycol)-octadecane (or “OPO”).

FIG. 5 includes images of results obtained from using in vitro testing of virus release from a representative polymer-based gel implant, wherein an adeno-associated virus (AAV) vector is released from polymer-based gel implant and retains activity as evidenced by transduction of HEK 293 cells in comparison to control samples wherein the AAV vector is delivered in a PBS buffer.

FIGS. 6A-6C are photographic images showing results after ex vivo administration of an OPO gel implant in pig eyes and demonstrating retinal adhesion and transition of the OPO gel implant to liquid phase at body temperature, wherein FIG. 6A shows the OPO gel implant administered using a soft-tipped cannula;

FIG. 6B shows the gel implant adheres to ex vivo pig retina; FIG. 6C shows that the gel implant transitions after 6 hours to a liquid phase;

FIGS. 7A-7C are photographic images showing results after in vivo administration of an OPO-AAV gel implant in primate eyes, wherein after two months after administration, release and transition was complete, retinas were healthy, and no adverse immune response or toxicity was noted as shown by FIG. 7A; FIGS. 7B and 7C show that GFP expression was observed in the primate fovea, under the area of OPO administration.

### DETAILED DESCRIPTION

### Overview of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment and may be applied to any embodiment disclosed.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

To facilitate review of the various embodiments of the disclosure, the following explanations of specific terms are provided. Certain functional group terms include a symbol “-” which is used to show how the defined functional group attaches to, or within, the compound to which it is bound. A person of ordinary skill in the art would recognize that the definitions provided below and the compounds and formulas included herein are not intended to include impermissible substitution patterns (e.g., methyl substituted with 5 different groups, and the like). Such impermissible substitution patterns are easily recognized by a person of ordinary skill in the art. In formulas and compounds disclosed herein, a hydrogen atom is present and completes any formal valency requirements (but may not necessarily be illustrated) wherever a functional group or other atom is not illustrated. For example, a phenyl ring that is drawn as

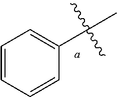 [SMILES: *c1ccccc1]

comprises a hydrogen atom attached to each carbon atom of the phenyl ring other than the “a” carbon, even though such hydrogen atoms are not illustrated. Any functional group disclosed herein and/or defined above can be substituted or unsubstituted, unless otherwise indicated herein.

Adeno-associated Virus (AAV): AAV is a small virus that infects humans and some other primate species. AAV is not currently known to cause disease and consequently the virus causes a very mild immune response. AAV can infect both dividing and non-dividing cells and may incorporate its genome into that of the host cell. The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. Rep is composed of four overlapping genes encoding Rep proteins required for the AAV life cycle, and Cap contains overlapping nucleotide sequences of capsid proteins: VP1, VP2 and VP3, which interact together to form a capsid of an icosahedral symmetry. For gene therapy, ITRs seem to be the only sequences required in cis next to the therapeutic gene: structural (cap) and packaging (rep) genes can be delivered in trans.

Age-related macular degeneration (AMD): A condition in which the cells of the macula (the central part of the retina) degenerate, resulting in loss of central visual acuity. AMD is the most common cause of irreversible loss of central vision and legal blindness in the elderly. It causes progressive damage to the macula, resulting in gradual loss of central vision. There are two forms, atrophic and neovascular macular degeneration. In atrophic degeneration (dry form), the tissues of the macula thin as photoreceptor cells disappear. There is currently no treatment for atrophic degeneration, though dietary supplements may help slow progression. In neovascular macular degeneration (wet form), abnormal blood vessels develop under the macula. These vessels may leak fluid and blood under the retina and eventually a mound of scar tissue develops under the retina. Central vision becomes washed out and loses detail, and straight lines may appear wavy. For neovascular macular degeneration there are some treatments available, including the use of medication injected directly into the eye (e.g., anti-VEGF therapy), laser therapy in combination with a targeting drug (e.g., photodynamic therapy) and brachytherapy. However, repeated treatments can cause complications leading to loss of vision.

Aliphatic: A hydrocarbon group having at least one carbon atom to 50 carbon atoms (C₁₋₅₀), such as one to 25 carbon atoms (C₁₋₂₅), or one to ten carbon atoms (C₁₋₁₀), and which includes alkanes (or alkyl), alkenes (or alkenyl), alkynes (or alkynyl), including cyclic versions thereof, and further including straight- and branched-chain arrangements, and all stereo and position isomers as well. Cyclic aliphatic groups comprising alkenes are distinct from aromatic groups.

Alkenyl: An unsaturated monovalent hydrocarbon having at least two carbon atoms to 50 carbon atoms (C₂₋₅₀), such as two to 25 carbon atoms (C₂₋₂₅), or two to ten carbon atoms (C₂₋₁₀), and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cycloalkenyl), cis, or trans (e.g., Eor Z). Cyclic alkenyl groups are distinct from aromatic groups.

Alkyl: A saturated monovalent hydrocarbon having at least one carbon atom to 50 carbon atoms (C₁₋₅₀), such as one to 25 carbon atoms (C₁₋₂₅), or one to ten carbon atoms (C₁₋₁₀), wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atoms to 50 carbon atoms (C₂₋₅₀), such as two to 25 carbon atoms (C₂₋₂₅), or two to ten carbon atoms (C₂₋₁₀), and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Aromatic: A cyclic, conjugated group or moiety of, unless specified otherwise, from 5 to 15 ring atoms having a single ring (e.g., phenyl) or multiple condensed rings in which at least one ring is aromatic (e.g., naphthyl, indolyl, or pyrazolopyridinyl); that is, at least one ring, and optionally multiple condensed rings, have a continuous, delocalized π-electron system. Typically, the number of out of plane π-electrons corresponds to the Hückel rule (4n+2). The point of attachment to the parent structure typically is through an aromatic portion of the condensed ring system. For example,

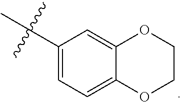 [SMILES: *c1ccc2c(c1)OCCO2]

However, in certain examples, context or express disclosure may indicate that the point of attachment is through a non-aromatic portion of the condensed ring system. For example,

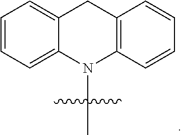 [SMILES: *N1c2ccccc2Cc2ccccc21]

An aromatic group or moiety may comprise only carbon atoms in the ring, such as in an aryl group or moiety, or it may comprise one or more ring carbon atoms and one or more ring heteroatoms comprising a lone pair of electrons (e.g., S, O, N, P, or Si), such as in a heteroaryl group or moiety. Aromatic groups may be substituted with one or more groups other than hydrogen, such as aliphatic, heteroaliphatic, haloaliphatic, haloheteroaliphatic, aromatic, or an organic functional group.

Aryl: An aromatic carbocyclic group comprising at least five carbon atoms to 15 carbon atoms (C₅-C₁₅), such as five to ten carbon atoms (C₅-C₁₀), having a single ring or multiple condensed rings, which condensed rings can or may not be aromatic provided that the point of attachment to a remaining position of the compounds disclosed herein is through an atom of the aromatic carbocyclic group. Aryl groups may be substituted with one or more groups other than hydrogen, such as aliphatic, heteroaliphatic, haloaliphatic, haloheteroaliphatic, aromatic, or an organic functional group.

Autoimmune retinopathy: Damage to the retina caused by autoantibodies to retinal proteins, which causes sudden and progressive loss of vision, leading to blindness. Autoimmune retinopathies include cancer-associated retinopathy (CAR), melanoma-associated retinopathy (MAR), autoimmune retinopathy (AR), and acute zonal occult outer retinopathy (AZOOR). Retinal proteins associated with autoimmune retinopathy include recoverin, carbonic anhydrase II, transducin-α, α-enolase, arrestin, aldolase, glyceraldehyde 3-phosphate dehydrogenase (GAPDH), tubby-like protein 1 (TULP1), heat shock protein 70, and photoreceptor cell-specific nuclear receptor.

Carbonyl: —C(O)—.

Clustered regularly interspaced short palindromic repeats (CRISPR) associated protein 9 (Cas9): An RNA-guided DNA endonuclease enzyme associated with the CRISPR (Clustered Regularly Interspersed Palindromic Repeats) adaptive immunity system in Streptococcus pyogenes, among other bacteria. Cas9 can cleave nearly any sequence complementary to the guide RNA. Includes Cas9 nucleic acid molecules and proteins. Cas9 sequences are publically available, for example from the GENBANK® sequence database (e.g., Accession Nos. NP_269215.1 and AKS40378.1 provide exemplary Cas9 protein sequences, while Accession No. NC_002737.2 provides an exemplary Cas9 nucleic acid sequence therein). One of ordinary skill in the art can identify additional Cas9 nucleic acid and protein sequences, including Cas9 variants.

Diabetic retinopathy: Damage to the retina that occurs as a complication of diabetes. Diabetic retinopathy is caused by changes in the blood vessels of the retina. There are four stages: 1) mild nonproliferative retinopathy, which includes occurrence of microaneurysms; 2) moderate nonproliferative retinopathy, which includes blockage of some vessels that feed the retina; 3) severe nonproliferative retinopathy, which includes more severe vessel blockage; and 4) proliferative retinopathy, which includes growth of abnormal blood vessels on the retina and the vitreous. Damage to the retina and/or vision loss occurs when these vessel leak or hemorrhage. Macular edema may also occur, particularly during the nonproliferative stages of the condition. Diabetic retinopathy is considered a subset of vascular retinopathy.

Gel Implant: A material capable of being implanted within an ocular region and that is a semi-solid, but is not a lyophilized solid. In gel implant embodiments disclosed herein, the gel implant comprises water and may have a viscous consistency or a soft, solid, or solid-like consistency. In some embodiments, the gel implant is in the form of a hydrated gel that comprises more than 10% water by weight.

Haloaliphatic: An aliphatic group wherein one or more hydrogen atoms, such as one to 10 hydrogen atoms, independently is replaced with a halogen atom, such as fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, silicon, boron, selenium, phosphorous, and oxidized forms thereof within the group. Alkoxy, ether, amino, disulfide, peroxy, and thioether groups are exemplary (but non-limiting) examples of heteroaliphatic.

Implanting (or Implantation): Inserting a polymer-based gel implant within an ocular region. The polymer-based gel implant does not need to be fixed in a position to be implanted, but in some embodiments it can become fixed via mucoadhesion. Implantation does not include implanting a solid, lyophilized form of the gel implant.

Leber congenital amaurosis (LCA): A rare inherited eye disease that appears at birth or in the first few months of life and primarily affects the retina. The presentation can vary because is it associated with multiple genes. However, it is characterized by characterized by nystagmus, photophobia, sluggish or absent pupillary response, and severe vision loss or blindness. The pupils, which usually expand and contract in response to the amount of light entering the eye, do not react normally to light. Instead, they expand and contract more slowly than normal, or they may not respond to light at all. Additionally, the clear front covering of the eye (the cornea) may be cone-shaped and abnormally thin, a condition known as keratoconus. A specific behavior called Franceschetti's oculo-digital sign is characteristic of Leber congenital amaurosis. This sign consists of poking, pressing, and rubbing the eyes with a knuckle or finger.

Ocular region: Any area of the eye, including the anterior and posterior segment of the eye, and which generally includes, but is not limited to, any functional (e.g., for vision) or structural tissues found in the eyeball, or tissues or cellular layers that partly or completely line the interior or exterior of the eyeball.

Ocular regions include the anterior chamber, the posterior chamber, the vitreous cavity, the choroid, the suprachoroidal space, the subretinal space, the conjunctiva, the subconjunctival space, the episcleral space, the intracorneal space, the epicorneal space, the sclera, the pars plana, surgically-induced avascular regions, the macula, and the retina.

Organic Functional Group: A functional group that may be provided by any combination of aliphatic, heteroaliphatic, aromatic, and/or haloaliphatic groups, or that may be selected from, but not limited to, aldehyde (i.e., —C(O)H); aroxy (i.e., —O-aromatic); acyl halide (i.e., —C(O)X, wherein X is a halogen, such as Br, F, I, or Cl); halogen; nitro (i.e., —NO₂); cyano (i.e., —CN); azide (i.e., —N₃); carboxyl (i.e., —C(O)OH); carboxylate (i.e., —C(O)O— or salts thereof, wherein the negative charge of the carboxylate group may be balanced with an M⁺ counterion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); amide (i.e., —C(O)NR^{a}R^{b} or —NR^{a}C(O)R^{b} wherein each of R^{a} and R^{b} independently is selected from hydrogen, aliphatic, heteroaliphatic, haloaliphatic, aromatic, or an organic functional group); ketone (i.e., —C(O)R^{a}, wherein R^{a} is selected from aliphatic, heteroaliphatic, haloaliphatic, aromatic, or an organic functional group); carbonate (i.e., —OC(O)OR^{a}, wherein R^{a} is selected from aliphatic, heteroaliphatic, haloaliphatic, aromatic, or an organic functional group); imine (i.e., —C(═NR^{a})R^{b} or —N═CR^{a}R^{b}, wherein R^{a} and R^{b} independently is selected from hydrogen, aliphatic, heteroaliphatic, haloaliphatic, aromatic, or an organic functional group); azo (i.e., —N═NR^{a} wherein R^{a} is hydrogen, aliphatic, heteroaliphatic, haloaliphatic, aromatic, or an organic functional group); carbamate (i.e., —OC(O)NR^{a}R^{b}, wherein each of R^{a} and R^{b} independently is selected from hydrogen, aliphatic, heteroaliphatic, haloaliphatic, aromatic, or an organic functional group); hydroxyl (i.e., —OH); thiol (i.e., —SH); sulfonyl (i.e., —SO₂R^{a}, wherein R^{a} is selected from hydrogen, aliphatic, heteroaliphatic, haloaliphatic, aromatic, or an organic functional group); sulfonate (i.e., —SO₃, wherein the negative charge of the sulfonate group may be balanced with an M⁺ counter ion, wherein M⁺ may be an alkali ion, such as K⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); oxime (i.e., —CR^{a}═NOH, wherein R^{a} is hydrogen, aliphatic, heteroaliphatic, haloaliphatic, aromatic, or an organic functional group); sulfonamide (i.e., —SO₂NR^{a}R^{b} or —N(R^{a})SO₂R^{b}, wherein each of R^{a} and R^{b} independently is selected from hydrogen, aliphatic, heteroaliphatic, haloaliphatic, aromatic, or an organic functional group); ester (i.e., —C(O)OR^{a} or —OC(O)R^{a}, wherein R^{a} is selected from aliphatic, heteroaliphatic, haloaliphatic, aromatic, or an organic functional group); thiocyanate (i.e., —S—CN or —N═C═S); thioketone (i.e., —C(S)R^{a} wherein R^{a} is selected from hydrogen, aliphatic, heteroaliphatic, haloaliphatic, aromatic, or an organic functional group); thiocarboxylic acid (i.e., —C(O)SH, or —C(S)OH); thioester (i.e., —C(O)SR^{a} or —C(S)OR^{a} wherein R^{a} is selected from hydrogen, aliphatic, heteroaliphatic, haloaliphatic, aromatic, or an organic functional group); dithiocarboxylic acid or ester (i.e., —C(S)SR^{a} wherein R^{a} is selected from hydrogen, aliphatic, heteroaliphatic, haloaliphatic, aromatic, or an organic functional group); phosphonate (i.e., —P(O)(OR^{a})₂, wherein each R^{a} independently is hydrogen, aliphatic, heteroaliphatic, haloaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); phosphate (i.e., —O—P(O)(OR^{a})₂, wherein each R^{a} independently is hydrogen, aliphatic, heteroaliphatic, haloaliphatic, aromatic, or an organic functional group; or wherein one or more R^{a} groups are not present and the phosphate group therefore has at least one negative charge, which can be balanced by a counterion, M⁺, wherein each M⁺ independently can be an alkali ion, such as K⁺, Na⁺, Li⁺; an ammonium ion, such as ⁺N(R^{b})₄ where R^{b} is H, aliphatic, heteroaliphatic, haloaliphatic, or aromatic; or an alkaline earth ion, such as [Ca²⁺]_{0.5}, [Mg²⁺]_{0.5}, or [Ba²⁺]_{0.5}); silyl ether (i.e., —OSiR^{a}R^{b}, wherein each of R^{a} and R^{b} independently is selected from hydrogen, aliphatic, heteroaliphatic, haloaliphatic, aromatic, or an organic functional group); sulfinyl (i.e., —S(O)R^{a}, wherein R^{a} is selected from hydrogen, aliphatic, heteroaliphatic, haloaliphatic, aromatic, or an organic functional group); thial (i.e., —C(S)H); or combinations thereof.

Pharmaceutically acceptable excipient: A substance, other than the therapeutic agent, that is included in a polymer-based gel implant. As used herein, an excipient typically is physically mixed with the polymer component and/or therapeutic agent of the polymer-based gel implant. An excipient can be used, for example, to dilute a therapeutic agent and/or to modify properties the polymer component and/or therapeutic agent of the polymer-based gel implant. Excipients can include, but are not limited to, antiadherents, binders, coatings, enteric coatings, disintegrants, flavorings, sweeteners, colorants, lubricants, glidants, sorbents, preservatives, carriers, or vehicles. Excipients may be starches and modified starches; cellulose and cellulose derivatives; saccharides and their derivatives, such as disaccharides, polysaccharides, and sugar alcohols; protein; synthetic polymers; crosslinked polymers; antioxidants; amino acids; or preservatives. Exemplary excipients include, but are not limited to, magnesium stearate, stearic acid, vegetable stearin, sucrose, lactose, starches, hydroxypropyl cellulose, hydroxypropyl methylcellulose, xylitol, sorbitol, maltitol, gelatin, polyvinylpyrrolidone (PVP), polyethyleneglycol (PEG), tocopheryl polyethylene glycol 1000 succinate (also known as vitamin E TPGS, or TPGS), carboxy methyl cellulose, dipalmitoyl phosphatidyl choline (DPPC), vitamin A, vitamin E, vitamin C, retinyl palmitate, selenium, cysteine, methionine, citric acid, sodium citrate, methyl paraben, propyl paraben, sugar, silica, talc, magnesium carbonate, sodium starch glycolate, tartrazine, aspartame, benzalkonium chloride, sesame oil, propyl gallate, sodium metabisulphite or lanolin.

Retinal degeneration: Deterioration of the retina, including progressive death of the photoreceptor cells of the retina or associated structures (such as retinal pigment epithelium). Retinal degeneration includes diseases or conditions such as retinitis pigmentosa, cone-rod dystrophy, macular degeneration (such as age-related macular degeneration and Stargardt-like macular degeneration), and maculopathies.

Retinal ganglion cell (RGC): A neuron located in the ganglion cell layer of the retina. RGCs receive neural inputs from amacrine cells and/or bipolar cells (which themselves receive neural input from photoreceptor cells). The axons of RGCs form the optic nerve, which transmits information from the retina to the brain.

Retinal responsiveness to light: The ability of one or more cells of the retina to respond to light, for example by producing an electrical signal and/or perception of a visual stimulus by a subject. Retinal response to light can be measured by detecting number, size, and/or frequency of electrical signals from the retina, for example by direct retinal recording (in vitro or in vivo), electroretinogram, or measuring visual evoked responses. Retinal response to light can also be measured by reporting of detection of a visual stimulus by a subject, for example wherein the subject closes a switch or presses a button when a visual stimulus is seen.

Retinitis pigmentosa (RP): A group of inherited retinal disorders that eventually lead to partial or complete blindness, characterized by progressive loss of photoreceptor cell function. Symptoms of RP include progressive peripheral vision loss and night vision problems (nyctalopia) that can eventually lead to central vision loss. RP is caused by mutations is over 100 different genes, and is both genotypically and phenotypically heterogeneous. Approximately 30% of RP cases are caused by a mutation in the rhodopsin gene. The pathophysiology of RP predominantly includes cell death of rod photoreceptors; however, some forms affect cone photoreceptors or the retinal pigment epithelium (RPE). Typical clinical manifestations include bone spicules, optic nerve waxy pallor, atrophy of the RPE in the mid periphery of the retina, retinal arteriolar attenuation, bull's eye maculopathy, and peripheral retinal atrophy.

Subject: Human and non-human subjects, including avian species and non-human mammals, such as non-human primates, companion animals (such as dogs and cats), livestock (such as ungulates and/or ruminants), as well as non-domesticated animals, such as the big cats.

Therapeutically Effective Amount: A quantity of a specified therapeutic agent sufficient to achieve a desired effect in a subject being treated with that therapeutic agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition. For example, a “therapeutically effective amount” may be a level or amount of agent needed to treat a retinal disease and/or retinopathy, or reduce or prevent retinal disease and/or retinopathy without causing significant negative or adverse side effects to the eye or a region of the eye.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform, or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Virus: Microscopic infectious organism that reproduces inside living cells. A virus consists essentially of a core of a single nucleic acid surrounded by a protein coat and has the ability to replicate only inside a living cell. “Viral replication” is the production of additional virus by the occurrence of at least one viral life cycle. Viral vectors are known in the art, and include, for example, adenovirus, AAV, lentivirus and herpes virus.

### INTRODUCTION

Until recently, there have been no effective treatments for retinal diseases and/or retinopathies. While certain gene therapies for retinal dystrophy have been developed, there is a significant need to develop new approaches for others forms of retinal degeneration. Also, the surgical approach used for certain treatments often are suboptimal in retinas with more significant structural alterations. As such, there is a need in the art for alternative treatments that can be adapted to various different administration techniques used for ocular treatment and that exhibit flexibility in terms of the particular cells to be targeted and/or the particular retina structure of a subject.

Disclosed herein are polymer-based gel implant embodiments that can be used to improve clinical outcomes and to extend the application of gene therapies to numerous retinopathies at various stages of disease. The disclosed polymer-based gel implant embodiments are able to target specific cell types, treat the macular area without damaging the remaining photoreceptors (a potential concern with sub-retinal injections in conditions where the retina is structurally compromised), exhibit efficient panretinal gene expression, and/or limit the inflammatory/immune responses associated with intravitreal injections. The polymer-based gel implant embodiments disclosed herein also permit a means for administering various therapeutic components while avoiding treatments or methods that might place stresses on the therapeutic agent, such as drying or lyophilizing the gel implant to provide a solid implant.

Polymer-based gel implant embodiments of the present disclosure exhibit good biocompatibility, particularly with the retina, and do not produce degradation products when in use. The polymer-based gel implants also are suitable for providing therapeutic agents to other areas of the eye, such as the fovea. Biocompatibility of a material used for retinal therapy has been a short-coming of treatments developed in the field prior to the present disclosure. Polymeric materials that may have been used in the art, which are safe for use in some parts of the eye, can be unsafe when used on or near the retina, which can be due at least in part to the buildup of degradation byproducts as the material undergoes successive biochemical cleavage of polymer chains (typically via hydrolysis) and dissolution of the resulting oligomeric or monomeric units.

Materials used in the art for intravitreally administered gene therapy also have exhibited a lack of control over vector localization. For example, controlled, consistent placement of nano- or microscale systems (such as solid particles, liposomes, and dendrimers) is difficult as these materials typically are distributed throughout the vitreous. And, conventional hydrogels and other non-particulate delivery methods currently used in the art tend to sink rather than diffuse. Diffuse materials in the vitreous also are subjected to higher rates of inactivation, particularly inactivation by neutralizing antibodies. The polymer-based gel implant embodiments of the present disclosure are better able to avoid the high rates of inactivation than other treatments that administer therapeutics/vectors by diffusing them through the vitreous.

Other drawbacks associated with existing particulate and non-particulate systems that are administered intravitreally include the requirement that the amount injected must be well above the desired virus titer in order to ensure sufficient retinal delivery and/or reflux of the injected solution is common and can lead to additional inconsistencies with the administered dose. In contrast, the polymer-based gel implant embodiments described herein can be pre-loaded with a therapeutic agent prior to administering the polymer-based gel implant, thereby providing a known and controllable amount of the therapeutic agent. In embodiments where the therapeutic agent is a vector, the polymer-based gel implant can be designed to contain a known titer of virus homogeneously distributed within polymer-based gel implant material on a per mass basis.

In some embodiments, the polymer-based gel implant can be used to deliver high efficiency vectors, such as adeno-associated virus (AAV) vectors, directly to the retina from the vitreous. Precise dosages of therapeutics and/or vectors can be administered using the disclosed polymer-based gel implant embodiments and the polymer-based gel implant can be directed to specific retinal locations and are flexible in that they can be specifically administered by depositing the gel implant such that it matches a particular retinal structure and/or geographic atrophy region of a subject. The disclosed polymer-based gel implant embodiments and methods of using the same provide the ability to target photoreceptors and RPE cells, which are the two main cell types involved in retinal degeneration. The polymer-based gel implant embodiments also can be used in combination with other retinal degeneration therapy, such as optogenetic therapy, gene transfer of rod-derived cone viability factor, CRISPR-Cas9 therapy, and the like.

### Polymer-Based Gel Implant Embodiments

Disclosed herein are embodiments of a polymer-based gel implant for use in treating retinal diseases and/or retinopathies. In particular disclosed embodiments, the polymer-based gel implant comprises a polymer component and a therapeutic agent. Each of these components of the polymer-based gel implant are described in more detail below.

The polymer component typically comprises one or more polymer unit species, wherein each polymer unit species can be the same or different as any other polymer unit species included in the polymer component. In some embodiments, the polymer component comprises a single polymer unit species. In some embodiments, the polymer component is a co-polymer, which comprises two polymer unit species that typically are structurally distinct from one another. In yet some other embodiments, the polymer component is a tri-block co-polymer, which can comprise three different polymer unit species or two different polymer species. In some embodiments, the polymer component comprises a polymer species unit that is bound to two end-capping groups. In such embodiments, the polymer component can have a formula A-B-A, wherein each A component independently is an end-capping group and the B component is a polymer unit species.

In some embodiments, both A components of the A-B-A triblock co-polymer are identical and in other embodiments the A components can be different (either in terms of structural identity, molecular weight, or the like). Representative classes and species of compounds that can be used as the polymer unit species of the polymer component are described below, as well as representative classes and species of end-capping groups.

In particular disclosed embodiments, the polymer component is a biocompatible bioerodible polymer. In yet additional embodiments, the polymer component is optically transparent or becomes optically transparent within a short time period after being implanted in an ocular region. The polymer component can include crosslinks among various polymer species units in the polymer and thus can form a crosslinked matrix. In particular embodiments, the polymer component is capable of undergoing different phase transitions upon hydration in an aqueous environment, such as the vitreous of the eye. Solely by way of example, the polymer component can transition from a gel phase to a liquid phase over a certain time period after being exposed to an aqueous environment. In some embodiments, tapered release can be achieved as the polymer component transitions from the gel phase to a more liquid phase, and complete release can occur as the polymer component transitions fully to the liquid phase. In particular disclosed embodiments, the therapeutic agent can be released as the concentration of the polymer component decreases below 25% (w/v).

In some embodiments, the polymer unit species can be selected from hydrocarbon polymers, heteroaliphatic polymers, carbonyl-containing polymers, haloaliphatic polymers, and saccharide-based polymers.

Representative hydrocarbon polymers can include, but are not limited to, polyalkylenes, such as polyethylene, polypropylene, polystyrene, or combinations thereof.

Representative heteroaliphatic polymers can include, but are not limited to, polyalkylene glycols, poloxamines, polyalkylene oxides, polyvinyl alcohols, polyvinyl ethers, polysiloxanes, polyvinyl esters, polyvinylpyrrolidone, poly(vinyl acetate), or any combinations thereof. In some embodiments, the heteroaliphatic polymer can be a polyethylene glycol polymer (PEG), or a polypropylene glycol polymer (PPG).

Representative haloaliphatic polymers can include, but are not limited to, polyvinyl halides, such as polyvinyl chloride, fluorinated polyethylene polymers, fluorinated polypropylene polymers, poly vinyl chloride polystyrene or any combinations thereof.

Representative carbonyl-containing polymers can include, but are not limited to, polyamides, polycarbonates, polyesters, polyalkylene terephthalates, polyurethanes, polyglycolides, polyhydroxyacids, polyhydroxyalkanoates. Exemplary carbonyl-containing polymers can include poly(methyl methacrylate), poly(ethylmethacrylate), poly(butylmethacrylate), poly(isobutylmethacrylate), poly(caprolactone), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), poly lactic acid, poly(lactic-co-glycolic acid), or any combinations thereof.

Representative saccharide-based polymers can include, but are not limited to, alkyl cellulose, hydroxyalkyl celluloses, cellulose ethers, cellulose esters, nitro celluloses, methyl cellulose, ethyl cellulose, hydroxypropyl cellulose, alginate, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, dextran, chitosan, or any combinations thereof.

Polymer component embodiments of the present disclosure also can exhibit mucoadhesive properties and thus can facilitate adhering polymer-based gel implant embodiments to the retina during hydration. For example, the mucoadhesive properties of the polymer component can promote adhering the polymer-based gel implant to glycoproteins present in the eye, particularly in the retina. In yet some additional embodiment, a polymer-based gel implant comprising a polymer component that does not exhibit mucoadhesive properties can be modified to comprise one or more mucoadhesive polymer additives.

Exemplary mucoadhesive polymer additives can include chitosan, hyaluronic acid, and the like. In some embodiments, retinal adhesion can be evaluated using washability measurements, wherein a final mass of the polymer-based gel implant is compared to the initial mass of the polymer-based gel implant and/or by determining mucin absorption on therapeutic agent-loaded gels to simulate adsorption to the mucin-like glycoproteins on the retina.

Representative end-capping groups can include hydrocarbon compounds, such as aliphatic groups, heteroaliphatic groups, aromatic groups, or combinations thereof. In some embodiments, the end-capping groups can be acyclic C₁₋₅₀aliphatic chains and/or acyclic C₁₋₅₀heteroaliphatic chains, which can be branched or un-branched; cyclic C₃₋₁₀aliphatic groups and/or cyclic C₁₋₅₀heteroaliphatic chains; aryl groups; heteroaryl groups; or combinations thereof.

In exemplary embodiments, the polymer component comprises a PEG polymer unit that is coupled at each end to an aliphatic end-capping group and thus has a structure satisfying the formula A-B-A. In particular embodiments, the aliphatic end-capping groups are octadecyl groups and thus each A component is an octadecyl group. In particular embodiments, the PEG polymer unit species is component B and it has a molecular weight of 10,000 g/mol and is —(OCH₂CH₂)₂₂₆O—. In such embodiments, the polymer component can have a formula C₁₈H₃₇—(OCH₂CH₂)₂₂₆O—C₁₈H₃₇. Other molecular weights of the PEG group can be used to control therapeutic agent release, as discussed below.

Therapeutic agents that can be included in the polymer-based gel implants can be selected from vectors, such as AAV vectors (e.g., AAV1, AAV2, AAV2-4YF, AAV2-4YFTV, AAV4, AAV6, AAV8, AAV8-2YF, AAV9, AAV9-2YF, AAVrh10, AAV11, AAV12, or the like; therapeutic drugs, such as anti-angiogenics (e.g., anti-VEGF antibodies or soluble receptors), fusion proteins (e.g., aflibercept), small molecules (e.g., ganciclovir), rod-derived cone viability factor (or other growth factors/proteins), naked DNA and/or RNA, chemotherapeutics (e.g., carboplatin or other chemotherapy for retinoblastoma); naturally and/or non-naturally occurring CRISPR-Cas9 systems comprising one or more AAV vectors; optogenetic therapeutic agents, such as an optogenetic actuator (e.g., channelrhodopsin, halorhodopsin, and/or archaerhodopsin), a promoter (e.g., CAMKIIα, Thy1, or the like), or combinations thereof, or one or more vectors that contain such actuators attached to a suitable promoter; or any combinations thereof. In yet additional embodiments, the polymer-based gel implant can comprise an additional drug delivery vehicle, such as a micelle, a dendrimer, a carbon nanotube, a liposome, a hydrogel particle, a protein nanoparticle, a polymer nano- or microsphere or any combinations thereof (and including pluralities of any such vehicles). In some embodiments, the vector is an AAV vector that is added at a concentration ranging from 1×10⁹ to 1×10¹² particles per mL.

In particular disclosed embodiments, the therapeutic component used in the polymer-based gel implant is a vector, such as an AAV vector (including recombinant AAV vectors). Particular vector embodiments are designed to infect retinal cells, photoreceptor (rod and/or cone) cells, retinal ganglion cells, RPE cells, Müller cells, retinal pigmented epithelial cells, bipolar cells, amacrine cells (including amacrine cells A and B), astrocytes, microglia, pericytes, vascular endothelium cells, horizontal cells, and other cells located in the ocular region and/or associated with the ocular region. In some embodiments, the vector can comprise a heterologous nucleic acid comprising a nucleotide sequence encoding a gene product, such as an interfering RNA (e.g., interfering RNA that decreases the level of apoptotic and/or angiogenic factors in a cell), an aptamer (e.g., aptamers active against vascular endothelial growth factor), a polypeptide (e.g., a polypeptide that enhances function of a retinal cell, such as the function of a rod or cone photoreceptor cell, a retinal ganglion cell, a bipolar cell, an amacrine cell, a Müller cell, a microglia cell, a pericyte cell, an RPE cell, a horizontal cell, a vascular endothelium cell, a retinal pigmented epithelial cell, or the like), a site-specific endonuclease (e.g., an endonuclease that provides for site-specific knock-down of gene function, such as knocking out an allele associated with a retinal disease), or any combinations thereof.

In particular embodiments of the disclosed polymer-based gel implant, the therapeutic agent is associated with the polymer component such that it is embedded in, dissolved in, dispersed in, adsorbed on, suspended in, or bound to the polymer component. The amount of the therapeutic agent included in the polymer-based gel implant can be determined based on a particular dosage that is to be achieved after implantation. In particular disclosed embodiments, a therapeutically effective amount of the therapeutic agent is provided. In some embodiments where the therapeutic agent is a vector, the dosage of the vector in the polymer-based gel implant is selected to match to a known titer of virus is used on a per mass basis. In some embodiments, the per mass basis of a vector loaded in a polymer-based gel implant embodiment can be determined by determining the total protein concentration released over time using, for example, a bicinchoninic acid assay. The result of any such protein assay provides an assessment of the maximum loading capacity of the polymer-based gel implant. In some embodiments, the vector can be provided in an amount that facilitates using a lowest feasible titer while still achieving efficient gene expression. Solely by way of example, the vector can be diluted to a desired multiplicity of infection (MOI) to lead to a particular percentage rate of transduction in a cell line of interest. Solely by way of example, a polymer-based gel implant can be prepared that comprises an AAV vector diluted to an MOI of 1500, which provides a concentration of the AAV vector that results in 50% of transduction in a cell line, such as in HEK 293 cells. In yet additional embodiments, the amount of the therapeutic agent included in the polymer-based gel implant can range from greater than 0 wt % to a maximum amount that can be included without deleteriously affective the phase transitions of the polymer-based gel implant. Factors that can be evaluated to determine suitable amounts of the therapeutic agent to include in the polymer-based gel implant can include osmotic pressure of the loaded therapeutic agent and the resulting viscosity of a suspension (if the therapeutic agent is a solid material) phase properties of the resulting polymer-based gel implant, and/or implantation/instillation capability.

The polymer component of the polymer-based gel implant can be modified to tune therapeutic agent release rate and/or the phase characteristics of the polymer-based gel implant. In some embodiments, the concentration of the polymer component included in the polymer-based gel implant (in terms of the resulting implant, not necessarily the initial concentration of the polymer component prior to implant formation) can be modified to influence the phase changes of the polymer-based gel implant, which, in some embodiments, can indirectly influence the release rate of the therapeutic agent. In some additional embodiments, the molecular weight of one or more of the polymer species units can be modified to increase or decrease the rate of therapeutic agent release. For example, the molecular weight of the one or more polymer species units can be increased so as to decrease hydration rates of the polymer component thereby decreasing therapeutic release rate. In yet additional embodiments, the amount of crosslinking (also referred to herein as crosslinking density) can be modified to increase or decrease the rate of therapeutic agent release. For example, the crosslinking density can be decreased to increase therapeutic agent release rate. In yet additional embodiments, therapeutic agent release rate can be decreased by incorporating a second therapeutic agent into the crosslinked matrix of the polymer-based gel implant.

The polymer-based gel implant is in gel form, particularly a transparent gel, when administered and can change phase to a liquid after a time period after implantation. Implanting the polymer-based gel implant in its gel form can facilitate slow release of therapeutic agents thereby enhancing protection from immune response and neutralizing antibody response. Furthermore, the gel form of the implant facilitates implantation without having to use more invasive surgical methods for implantation, such as cutting and other undesirable techniques needed to implant a solid implant. And, the polymer-based gel implant easily transitions to a liquid when in the eye and thus no surgical removal is needed. In particular disclosed embodiments, the polymer-based gel implant is transparent when administered to the subject and remains transparent even as it changes to a liquid phase. Such transparency facilitates its use in the eyes of subjects particularly in the retina as subjects are not visually impaired or irritated by the presence of the implant. In particular embodiments, the polymer-based gel implant is sufficiently transparent that it can be used for focal epiretinal implantation.

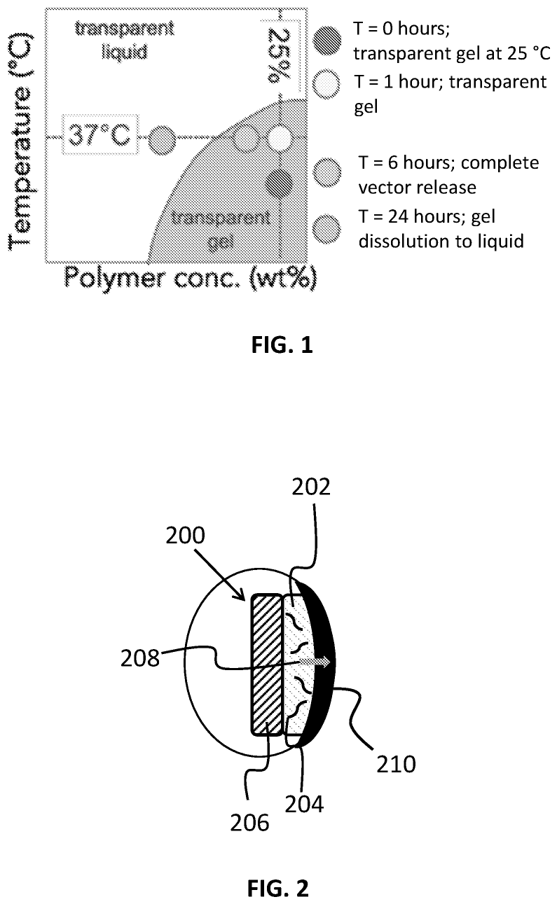

A representative phase diagram of a particular polymer-based gel implant of the present disclosure is illustrated in FIG. 1. As can be seen in FIG. 1, the polymer-based gel implant is in the form of a transparent gel at 25% (wt/v) polymer and at 25° C. The polymer-based gel implant is not in the form of a lyophilized solid when implanted. The polymer-based gel implant is hydrated such that it comprises at least some water, most typically more than 10% (w/v) water and, in some embodiments, at least 25% w/v water. In some embodiments, the amount of water included in the polymer-based gel implant ranges from greater than 10% to 50% w/v water, or greater than 10% to 40% w/v water, or greater than 10% to 30% w/v, or greater than 10% to 25% w/v, or greater than 10% w/v to 20% w/v, or greater than 10% w/v to 15% w/v water. In particular embodiments, the amount of water is 11%, 12%, 13%, 14%, 15%, 16%, 17%, 18%, 19%, 20%, 21%, 22%, 23%, 24%, or 25% w/v. As temperature increases, the gel remains transparent and in gel form until it begins to become hydrated and the polymer concentration decreases below 25% (w/v). No initial hydration period is needed, whereas if the gel were administered as a solid, lyophilized form of the implant that is water-free or substantially water-free, a hydration period would be needed. At 37° C., the gel will begin to hydrate and will release the therapeutic agent (e.g., a vector). The gel will eventually become hydrated to the point that it transitions to a liquid form. Both the gel and the liquid forms are transparent. Hydration of the polymer-based gel implant can thus affect polymer component concentration, which thereby controls vector release because, as water infiltrates the matrix of the polymer component, this allows for diffusion of therapeutic agent out of the polymer-based gel implant. During this therapeutic agent release phase, the polymer-based gel implant typically remains in the form of a hydrated gel, resulting in a higher level of control over the rate of vector release throughout an initial post-implantation period (e.g., a 6-hour post-implantation period as indicated by FIG. 1). After this post-implantation period (e.g., at 24 hours or more, such as shown in FIG. 1), the polymer component absorbs enough water to effectively reduce the polymer concentration such that the gel implant can shift to a liquid form. This final liquid form of the polymer component is transparent, biocompatible, and stable. In particular disclosed embodiments, the liquid form of the polymer component does not degrade and thus does not have to be affirmatively removed and also does not result in any toxicity within the ocular region. The transmittance of the gel across the visible light spectrum in its final hydrated form is 88-90% direct transmittance across 350-750 nm, indicating that the polymer-based gel implant is suitable for ocular implantation as this is substantially similar to the transmission window of the human vitreous (as well as other animals' vitreous humor). Further, in particular embodiments, the ¹H-NMR spectrum of an exemplary material exhibited the expected degree of substitution for polyethylene glycol along with sufficient resolution and separation for subsequent analyses, evidencing the ability to provide pure implant products with no evidence of toxic byproducts or reagents.

In some embodiments, because the polymer-based gel implant is a gel and thus has a soft, often pliable and/or liquid-type consistency, it is flexible and capable of adapting to the space in which it will be placed (e.g., it can adopt shapes or fill-in/occupy a desired space) when administered. The polymer-based gel implant also is capable of being added into a syringe for administration via injection. This ability to administer the polymer-based gel implant via syringe such that it can be added at any desired location in the ocular region facilitates the ability to administer the implant without invasive surgical techniques and with minimal patient discomfort. In particular embodiments, the polymer-based gel implant is applied on a surface of the retina and exhibits adherence to the retina.

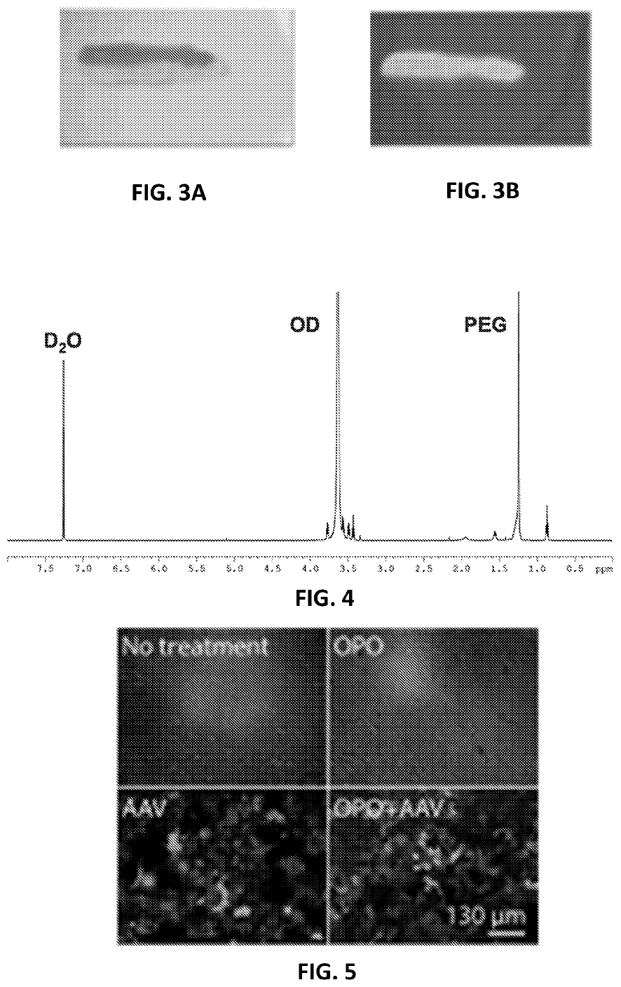

In yet additional embodiments, the polymer-based gel implant can further comprise a semi-permeable or fully impermeable backing layer material. In such embodiments, the backing layer material facilitates unidirectional diffusion of any therapeutic agent present in the polymer-based gel implant. As such, therapeutic agent release can be directed toward a particular region of the eye (e.g., the retina) and away from, for example, the vitreous body. The backing layer material can be provided as a separate material that can form a layer on the polymer-based gel implant and can be formed from a polymer unit species disclosed herein that can be the same or different (in terms of chemical identity, molecular weight, crosslink content, and/or concentration) as the polymer component of the polymer-based gel implant. In embodiments comprising a backing layer material, the lower density region (that is, a region of the polymer-based gel implant that is not adjacent to the backing layer material) can be loaded with the therapeutic agent and a higher density region can be free of any therapeutic agent. A schematic illustration of a polymer-based gel implant comprising a backing layer material is illustrated in FIG. 2. According to the embodiment shown in FIG. 2, polymer-based gel implant 200 comprises polymer component 202, which is in the form of a hydrated gel and in which therapeutic agent 204 is suspended, and further comprises backing layer material 206. Arrow 208 represents the direction of therapeutic agent release and, as illustrated in FIG. 2, backing layer material 206 prevents delivery of therapeutic agent 204 into the vitreous and thereby promotes delivery solely to retina 210. FIGS. 3A and 3B are images of an exemplary embodiment of a polymer-based gel implant that has been modified to comprise a backing layer material, which as can be seen from the images, facilitates fluorescein isothiocyanate (FITC) diffusion from the gel implant in a specific direction and prevents FITC diffusion into or past the backing layer material. In embodiments wherein the polymer-based gel implant is to be administered with a backing layer material, the gel implant and the backing layer material may be injected from the same syringe by using a dual-chamber syringe, wherein one chamber of the syringe houses the polymer-based gel implant material and the other chamber of the syringe houses the backing layer material. Such a device can be used to apply a layer of the polymer-based gel implant and a layer of the backing layer material.

### Methods of Using Polymer-Based Gel Implant Embodiments

Disclosed herein are embodiments of a method of using the polymer-based gel implant embodiments of the present disclosure. In some embodiments, the method comprises providing a polymer-based gel implant embodiment and implanting the polymer-based gel implant embodiment in a subject and particularly in an ocular region. In some embodiments, the polymer-based gel implant is implanted at or near the retina of the subject. In some additional embodiments, the polymer-based gel implant is implanted at or near the fovea of the subject. The polymer-based gel implant can be implanted using any suitable method for positioning the polymer-based gel implant on or near the desired region of a subject's eye. In some embodiments, the polymer-based gel implant is administered using a syringe or other suitable implantation device/technique. In some embodiments, the syringe can be dual-chamber syringe or a single-chamber syringe. In some independent embodiments, a superotemporal port or cannula can be used. In some embodiments, the polymer-based gel implant can be implanted via intravitreal injection. In yet other embodiments, the polymer-based gel implant can be implanted via subretinal or epiretinal injection. Embodiments of the method wherein the polymer-based gel implant is administered using intravitreal injection can further comprise performing a partial (wherein less than substantially all of the vitreous is removed) or full vitrectomy (wherein substantially all of the vitreous is removed). In an independent embodiment, the method does not comprise removing the polymer-based gel implant or any degradation product formed therefrom. The polymer-based gel implant is in the form of a hydrated gel when implanted and is not a lyophilized solid.

In particular disclosed embodiments, the polymer-based gel implant is used to deliver a retinal gene therapy to a subject's retina and/or fovea. In such embodiments, the therapeutic agent typically is a vector (or other gene-related therapy disclosed herein). The vector can be an AAV vector in some particular embodiments. Once implanted, the polymer-based gel implant can change phases from hydrated gel to liquid as discussed herein to facilitate vector release such that the vector can infect any targeted cells and interact with the cells (e.g., promote and/or stimulate cell growth, or inhibit and/or prevent cell growth).

The polymer-based gel implant embodiments of the present disclosure can be used to treat retinal diseases, retinopathies, and other ocular diseases in which the retina is involved. In yet additional embodiments, the polymer-based gel implant can be used to improve retinal responsiveness to light. In some embodiments, the polymer-based gel implant can be used to treat any one or more of the following retinal disorders/diseases: central retinal vein occlusion, diabetic retinopathy (including proliferative diabetic retinopathy), proliferative vitreoretinopathy (PVR), retinal arterial occlusive disease, retinal detachment, uveitic retinal disease, non-retinopathy diabetic retinal dysfunction, retinoschisis, retinitis pigmentosa (e.g., X-linked retinitis pigmentosa), epiretinal membrane disorders, radiation retinopathy, retinal vein occlusion, chorioretinal degeneration, retinopathy of prematurity, acute macular neuroretinopathy, and any combinations thereof. In yet additional embodiments, the polymer-based gel implant can be used to treat one or more of the following ocular diseases/disorders: sympathetic opthalmia, Vogt Koyanagi-Harada syndrome, uveal diffusion, a posterior ocular condition (e.g., a condition caused by or influenced by an ocular laser treatment), posterior ocular conditions (e.g., conditions caused by or influenced by a photodynamic therapy), photocoagulation, branch anterior ischemic optic neuropathy, glaucoma, Usher syndrome, cone-rod dystrophy, Stargardt disease, inherited macular degeneration, Leber congenital amaurosis (e.g., RPE65-LCA2), congenital stationary night blindness, choroideremia, Bardet-Biedl syndrome, macular telangiectasia, Leber's hereditary optic neuropathy, and disorders of color vision (e.g., achromatopsia, protanopia, deuteranopia, and tritanopia), Behcet's disease, choroidal neovascularization, diabetic uveitis, histoplasmosis, macular degeneration (e.g., acute macular degeneration, non-exudative age related macular degeneration, and exudative age related macular degeneration), edema (e.g., macular edema, cystoid macular edema, diabetic macular edema, or combinations thereof), multifocal choroiditis, ocular trauma (e.g., trauma that affects a posterior ocular site or location), ocular tumors, and any combinations thereof. In particular disclosed embodiments, the polymer-based gel implant is not used as a vitreous humor replacement gel.

In some embodiments, disease-relevant canine models are used to evaluate efficacy of the disclosed polymer-based gel implant embodiments. Canine models are known in the art to be appropriate models for validating retinal gene therapies for diseases that affect cells of the ocular region, such as RPE (e.g., RPE65-LCA; BEST1-bestrophinopathies), and photoreceptors (e.g., CNGB3-ACHM, RPGR-XLRP, PDE6B-arRP, RPGRIP1-CRD, RHO-adRP, CNGB1-arRP, NPHP5-LCA). rAAV serotypes and cell-specific promoters have been shown to enable targeting these retinal populations and they show similar tropism and transduction activity in the human eye. In addition, the large volume of the canine eye, with diseases that affect the newly discovered cone-enriched fovea-like area, provides a model system to evaluate the delivery of doses susceptible to be used for targeting the human foveo-macular region. Also, although the canine retina is devoid of foveal pit, a “canine fovea-like” area within the center of the cone enriched canine area centralis has been identified. This −100 μm diameter region has a peak density of cones that is similar to that found in the human fovea and is the only area of the canine retina where multiple (˜3) rows of cone somatas can be found, and where cones have an elongated “rod-like” appearance. In addition, dogs with mutations in two different genes (BEST1, RPGR), that cause inherited maculopathies in humans, develop earliest disease at this newly-identified canine fovea-like area, which makes the canine area centralis and its fovea-like area in particular, a suitable model system to study delivery of therapeutic agents to the macular area.

In particular embodiments, the XLPRA2 dog, which carries a frameshift mutation in exon ORF15 of RPGR, is used for evaluating the efficacy of the disclosed polymer-based gel implants. The XLPRA2 dog has been extensively characterized and found to closely recapitulate one of the phenotypes within the human disease spectrum that shows regional predilection for the central retina. Also, in the XLPRA2/RPGR mutant dog, early photoreceptor disease along the visual streak has been found to begin and progress more severely within the fovea-like area thus making it a suitable model system to test and validate therapeutic strategies aimed at targeting via subretinal or intravitreal routes photoreceptors the human foveo-macula.

In additional embodiments, the RPE65 dog can be used as a model of RPE65-LCA2 to assess focal or pan-retinal targeting of the retinal pigment epithelium. The canine model of RPE65-LCA is well-characterized. Visual impairment in RPE65 deficient dogs is caused by a homozygous 4-bp deletion in RPE65 resulting in a frameshift and a premature stop codon which truncates the protein. The disorder is characterized by congenital night blindness with various degrees of visual impairment under photopic illumination. Histologically retinas show prominent RPE inclusions and loss of S cones at an early age with progressive degeneration of rods and L/M cones later in life. More recently, it was shown in a colony of RPE65 dogs from Michigan State University that early-onset severe photoreceptor degeneration occurs in a specific region of the area centralis, thus sharing some phenotypic similarities with a subset of RPE65-LCA2 patients that exhibit early central cone loss. The RPE65 dog model can therefore be used to refine polymer-based gel implant delivery of therapeutic agents to focal regions, such as the fovea-like area or to more extended retinal surfaces and thus can be used to assess utility in other RPE diseases (such, as Best Vitelliform Macular Dystrophy, or MERTK RP).

### Methods of Making Polymer-Based Gel Implant Embodiments

Also disclosed herein are embodiments of making the polymer-based gel implant of the present disclosure. In some embodiments, the polymer-based gel implant is made by combining a therapeutic agent with a polymer component. In some embodiments, the therapeutic agent is embedded in, dissolved in, dispersed in, adsorbed on, suspended in, or bound to the polymer component. In exemplary embodiments, the therapeutic agent is suspended in the polymer component. In some embodiments, the therapeutic agent is a vector that is added at a concentration ranging from 1×10⁹ to 1×10¹² particles per mL. The polymer component, when combined with the therapeutic agent, typically is in the form of a hydrated gel. In some such embodiments, the polymer-based gel implant can consist of, or consists essentially of, the polymer component, the therapeutic agent, water, and, optionally, a pharmaceutically acceptable excipient. In some embodiments, the method can further comprise sterilizing the polymer-based gel implant, such as by using a terminal sterilization technique wherein the gel implant is subjected to gamma irradiation. In such embodiments, no loss in material mass or gross changes in appearance occur. In some embodiments, excipients also may be included in the polymer-based gel implant to prevent any aggregation of the therapeutic agent included therein. Such excipients can be selected from any of the pharmaceutically-acceptable excipients described herein.

In some embodiments, the polymer-based gel implant has a viscosity or physical characteristics that facilitate its ability to conform to a desired shape and/or size so as to match a particular geographic atrophy region of a subject's eye. In some embodiments, the viscosity of the polymer-based gel implant can be modified to have more less crosslinking within the polymer component to thereby modify its physical properties. In yet other embodiments, pharmaceutically acceptable excipients can be included so as to control the viscosity of the gel. In yet additional embodiments, more water can be added to the polymer component so as to decrease its viscosity. The tuneability of the gel implant facilitates near-infinite customization, including the ability of the gel implant to adopt a particular desired curvature to increase contact with the retina, such as when implanted from the intravitreal approach. In some embodiments, multiple injections of the polymer-based gel implant can be used to increase the surface area of coverage obtained with the polymer-based gel implant.

In yet additional embodiments, a polymer-based gel implant comprising a backing layer material can be made. In such embodiments, the polymer-based gel implant and the backing layer material can be made separately and then administered together, such as via a dual-chamber syringe. In other such embodiments, the polymer-based gel implant and the backing layer material can be made separately, combined, and then injected. In some embodiments, the polymer-based gel implant can be made to comprise a particular polymer component species and/or degree of cross-linking amongst the polymer component. The backing layer material can be made to comprise a different polymer component species from that of the polymer-based gel implant and/or to have a different degree of cross-linking as compared to the polymer-based component of the polymer-based gel implant. In particular embodiments, the backing layer material is made to have a higher density (e.g., such as by using a higher molecular weight polymer component and/or by providing a more densely cross-linked polymer component) than the polymer-based gel implant such that any therapeutic agent included in the polymer-based gel implant is not able to pass through the backing layer material and thus is unidirectionally dispersed from the polymer-based gel implant.

### Overview of Several Embodiments

Disclosed herein are embodiments of a polymer-based gel implant, comprising: a polymer component comprising one or more polymer species units, wherein the polymer component is capable of absorbing water such that the polymer component transitions from a gel phase to a liquid phase as a concentration of the polymer component in the polymer-based gel implant decreases; and a therapeutic agent suspended in the polymer component; wherein the polymer-based gel implant is a gel at ambient temperature and comprises water, optionally more than 10% (w/v) water.

In any or all of the above embodiments, the polymer component has a structure satisfying a formula A-B-A, wherein B is a polymer species unit and each A independent is an end capping group attached to each end of the polymer species unit.

In any or all of the above embodiments, each end capping groups is an aliphatic group.

In any or all of the above embodiments, the polymer species unit is a polyalkylene oxide.

In any or all of the above embodiments, the polymer component is octadecane-poly(ethylene glycol)-octadecane.

In any or all of the above embodiments, the therapeutic agent is selected from a vector, a pharmaceutical drug, an optogenetic therapeutic agent, a naturally and/or non-naturally occurring CRISPR-Cas9 system, or any combination thereof.

In any or all of the above embodiments, the vector is an AAV vector, a recombinant AAV vector, or any combination thereof.

In any or all of the above embodiments, the vector is capable of infecting retinal cells, photoreceptor (rod and/or cone) cells, retinal ganglion cells, RPE cells, Müller cells, retinal pigmented epithelial cells, bipolar cells, amacrine cells, astrocytes, microglia, pericytes, vascular endothelium cells, horizontal cells, and other cells located in the ocular region.

In any or all of the above embodiments, the therapeutic agent is suspended in the polymer component, which is in gel form.

In any or all of the above embodiments, the polymer-based gel implant comprises a backing layer material that facilitates unidirectional delivery of the therapeutic agent from the polymer component such that the therapeutic agent does not pass through the backing layer material.

In any or all of the above embodiments, the backing layer material comprises one or more polymer species units that are the same as the one or more polymer species units of the polymer component and wherein the one or more polymer species units of the backing layer have a different number of crosslinks as compared to the one or more polymer species units of the polymer component.

In any or all of the above embodiments, the backing layer material comprises one or more polymer unit species that does not absorb water.

In any or all of the above embodiments, the polymer-based gel implant becomes transparent upon exposure to an aqueous environment.

In any or all of the above embodiments, the polymer component is octadecane-poly(ethylene glycol)-octadecane and the therapeutic agent is an AAV vector.

Also disclosed herein are embodiments of a method, comprising: providing a polymer-based gel implant of any or all of the above implant embodiments; and implanting the polymer-based gel implant into an ocular region of a subject.

In any or all of the above embodiments, implanting is performed via injection.

In any or all of the above embodiments, the injection is an intravitreal injection, a subretinal injection, or a combination thereof.

In any or all of the above embodiments, the method further comprises performing a partial or full vitrectomy.

In any or all of the above embodiments, the method does not comprise removing the polymer-based gel implant or any degradation product formed therefrom.

Also disclosed herein are embodiments of a method, comprising treating a retinal disease and/or a retinopathy by implanting the polymer-based gel implant according to any or all of the above implant embodiments in an ocular region of a subject having, or capable of developing, the retinal disease and/or retinopathy.

In any or all of the above embodiments, the retinal disease and/or a retinopathy is selected from central retinal vein occlusion, diabetic retinopathy, proliferative vitreoretinopathy, retinal arterial occlusive disease, retinal detachment, uveitic retinal disease, non-retinopathy diabetic retinal dysfunction, retinoschisis, retinitis pigmentosa, epiretinal membrane disorders, radiation retinopathy, retinal vein occlusion, chorioretinal degeneration, retinopathy of prematurity, acute macular neuroretinopathy, sympathetic opthalmia, Vogt Koyanagi-Harada syndrome, uveal diffusion, a posterior ocular condition, posterior ocular conditions, photocoagulation, branch anterior ischemic optic neuropathy, glaucoma, Usher syndrome, cone-rod dystrophy, Stargardt disease, inherited macular degeneration, Leber congenital amaurosis, congenital stationary night blindness, choroideremia, Bardet-Biedl syndrome, macular telangiectasia, Leber's hereditary optic neuropathy, and disorders of color vision, Behcet's disease, choroidal neovascularization, diabetic uveitis, histoplasmosis, macular degeneration, edema, multifocal choroiditis, ocular trauma, ocular tumors, and any combinations thereof.

In any or all of the above embodiments, the retinal disease is RPE65-LCA2 or X-linked retinitis pigmentosa.

Also disclosed herein are embodiments of a method of making a polymer-based gel implant according to any or all of the above embodiments, comprising: hydrating the polymer component by combining it with water to provide a hydrated polymer component; and combining the hydrated polymer component and the therapeutic agent to provide the polymer-based gel implant.

### EXAMPLES

### Example 1

In this example, an exemplary polymer component was made. A polyethylene glycol (PEG)-n-octadecane copolymer was synthesized under inert nitrogen atmospheric conditions using a Schlenk line and oven dried glassware. Approximately 1 molar equivalent or 50 g of PEG (10 kDa, Sigma) was added to a 1-L 3-neck round bottom flask and dissolved in 300 ml of 1,4-dioxane (dry). Temperature was monitored continuously in one arm and nitrogen was continuously flushed in another, leaving the third for additions. The mixture was heated to 70° C. using a water bath. Once the PEG was fully dissolved, the reaction vessel was cooled to room temperature again using a water bath. Approximately 20 molar equivalents or 4.05 g of sodium hydride (60% 23.99 g/mol, Aldrich) was added in 4 aliquots and stirred for 1 hour. After 20 minutes the reaction mixture solidified and the water bath was heated to 40-45° C. to redissolve the solid material. Next, 20 molar equivalents or 34.2 ml of >97% 1-bromooctadecane (333.39 g/mol, Aldrich) was added dropwise, resulting in a slightly opaque yellow-orange solution. The reaction mixture was stirred at room temperature for 20 hours followed by concentration of the crude reaction mixture under vacuum, yielding a dark yellow-brown oil. The crude oil was dissolved in 200 ml of methylene chloride and extracted with 1.0 M HCl. The pH of the aqueous phase was 1.0. A persistent emulsion was observed throughout the extraction.

The organic phase was dried with magnesium sulfate, filtered via vacuum filtration, and once again concentrated under vacuum. The concentrate was a transparent yellow-orange oil, which was then purified via column chromatography using diethyl ether and methylene chloride. The silica gel column was prepared using diethyl ether and the crude product was loaded on the column. The desired end product, OD-PEG-OD (OPO), precipitated on top of the column. Residual bromooctadecane and its elimination product were eluted using diethyl ether. The final OPO polymer component was isolated via elution with methylene chloride as a white powder with a final yield of approximately 30%. The OPO polymer component was then rehydrated by adding water to provide a hydrated gel.

FIG. 4 shows the proton nuclear magnetic resonance (¹H-NMR) spectrum of the resulting polymer component. A weighed sample of the dry OPO material was dissolved in ethanol (1 mL) than pre-injected into the volumetric tube via the septum cap. The sample rested for a minimum of 1 hour and a maximum of 24 hours. A 500 ul aliquot was injected into a standard NMR tube previously flushed with N₂. The spectrum was acquired for a range of 120-256 scans and plotted. For each sample, one full 0-10 ppm spectrum was plotted alongside two expansions of the relevant region for integration; thus, the data obtained were for three separate integrations of the spectrum. The spectrum shown in FIG. 4 exhibits the expected degree of substitution for PEG along with sufficient resolution and separation for subsequent analyses. And, this spectrum demonstrates the ability to consistently synthesize a pure product as the product is free of contaminating reagents.

### Example 2

In this example, a polymer-based gel implant is made. AAV is suspended in the hydrated polymer component of Example 1 to provide a polymer-based gel implant embodiment. In some examples, ˜50 μL of 1×10¹² vg/mL titer vector is added to about 500 μL of hydrated polymer. Terminal sterilization of the polymer-based gel implant is achieved through gamma irradiation with minimal loss in material mass and no gross changes in appearance.

### Example 3

In this example, the fully characterized OPO gel implant is tested in benchtop simulations of in vivo use. Release of fluorescent nanoparticles as a surrogate for AAV are to be quantified over time from the OPO gel implant. A 25% (w/v) OD-PEG-OD gel containing 50 nm diameter nanoparticles is prepared by dissolving 250 mg of OD-PEG-OD in 1 ml of an aqueous solution of 50 nm Fluoresbrite Plain YG nanoparticles with a concentration of 3.64×10¹⁴ particles/ml. The gel material is then exposed to physiological conditions, e.g., DPBS (pH=7.4), using a stir rate of 8 RPM at 37° C. Release media in triplicate samples are collected every 30 minutes for 3 hours and thereafter every hour up to 6 hours followed by every 6 hours until reaching 24 hours. Nanoparticle concentrations in 250 μl release media aliquots are determined using UV-Vis spectroscopy, where emission intensities at 510 nm are measured and compared to a previously validated standard curve. A “burst” release of nanoparticles over 6 hours can be observed, with a lower amount of nanoparticles released over the remaining 18 hours. This should correspond well with the desired in vivo AAV release behavior, namely that the majority of AAV would be released before the gel implant has absorbed sufficient water to transition the gel fully to the liquid phase. Adjustments to AAV release can be achieved by modulating the porosity of the gel network by increasing or decreasing crosslinking density (to slow down or speed up AAV release, respectively). The fluorescent OPO gel implants are next placed on the retina through the vitreous in an ex vivo bovine eye experimental setup. Freshly enucleated, never frozen eyes (Pel-Freez Biologicals) are used immediately for these studies. The OPO gel implant is clearly observed throughout the study, along with the phase change and spreading of the fluorescent nanoparticles contained therein. The retinal adhesive properties of the OPO implant are preliminarily confirmed as there should be no movement of the OPO implant over time.

### Example 4

To test the ability of OPO-encapsulated AAV to be released and subsequently infect cells, a series of in vitro assays can be performed using AAV diluted to a MOI (multiplicity of infection) of 1500, a concentration determined to lead to ˜50% rate of transduction in HEK 293 cells in vitro. The virus is either suspended in PBS or loaded into the OPO gel implant. Virus is then either pipetted into one well of a 6 well plate, or the OPO gel implant is placed in a small cage suspended above the cells. The number of infected cells is equal in wells treated with PBS-diluted or OPO gel-embedded virus, indicating complete release of virus and no change in infectivity of the virus (see FIG. 5). These results suggest that the AAV particles are fully released and maintain activity. Assays also can incorporate primary cultures of RPE cells.

### Example 5

In this example, freshly enucleated rabbit eyes, which are closer in size and anatomy to human eyes than bovine eyes, and freshly obtained ex vivo human eyes are used to establish implantation procedures for the polymer-based gel implant of the present disclosure, followed by validation in canines. Polymer-based gel implants are loaded into a syringe and, once the instrument is positioned in front of the target area, are pushed to the desired location. Peri-operative OCT allows for checking the proper placement. The contribution of vitreous to AAV diffusion can be evaluated using fluorescent nanoparticle surrogates. In particular embodiments, vectors that promote expression of GFP are used in the polymer-based gel implant. Such embodiments can be implanted in vivo in WT dogs, which are kept under dorsal recumbency immediately after implantation for a suitable period of time for the polymer-based gel implant to release its AAV load and fully hydrate to liquid form.

### Example 6

In this example, the directionality of particle movement using fluorescent nanoparticles in a transwell assay can be assessed to evaluate performance of a polymer-based gel implant embodiment that is used in combination with a backing layer. The gel is exposed to water and elution of nanoparticles is simultaneously monitored. Particle elution over 24 hours is determined. These results are qualitatively confirmed using fluorescence microscopy of the polymer-based gel implants in solution. Exemplary results are shown in FIGS. 3A (prior to water exposure) and 3B (after water exposure).

### Example 7

In this example, nine male XLPRA2/RPGR mutant dogs are transplanted at the time of disease onset (˜6 weeks of age) with a 2 mm diameter OPO gel implant loaded with AAV-RPGR viral particles at one of three concentrations (1×, 10×, and 100×) predetermined based on results conducted in WT dogs. Dogs are randomly allocated to one of the three treatment groups (n=3 dogs/group). In all treatment groups, dogs have their contralateral eye implanted with the same type of OPO gel implant loaded with an AAV-GFP construct (same three doses as for AAV-RPGR). This negative control allows to verify that the AAV tropism towards canine photoreceptors (established in WT dogs) is retained in diseased/mutant retinas.

The AAV-RPGR construct comprises an AAV plasmid containing the same human stabilized RPGR cDNA sequence used in canine proof of concept studies. Unless a more potent promoter capable of driving transgene expression to both rods and cones is validated in non-human primate (NHP) and WT dogs' retinas, the human 292-nt portion of the human GRK1 promoter is used, as this promoter has been shown to be effective at turning on transgene expression in both classes of photoreceptors in dogs and in NHPs. The AAV plasmid is packaged in an AAV capsid variant that can efficiently target rods and cones in both NHPs and WT dogs. After epiretinal implantation, the dogs are followed for 18 weeks. Weekly ophthalmic examinations including fundus photography are performed to monitor ocular tolerability to the polymer-based gel implants. Longitudinal assessment of GFP expression (in the contralateral eyes), and structural integrity of the retinas is conducted by non-invasive csLO/OCT imaging (Spectralis HRA/OCT2) at pre-implantation (˜6 weeks of age=baseline), 12 weeks of age, and before termination at 24 weeks of age. In depth qualitative and quantitative analysis of ONL thickness is conducted after semi-manual segmentation of individual longitudinal reflectivity profiles from overlapping (30°×20°) raster OCT scans. Topographical maps of ONL thickness are examined to determine whether ONL rescue is seen in the area corresponding to the polymer-based gel implant implantation. The dimension of the region of rescue in comparison to that of the polymer-based gel implant will inform on the extent of potential tangential diffusion of AAV out of the polymer-based gel implant, and its ability to treat or not an area larger than that of the polymer-based gel implant.

Full field ERG is used to assess any functional recovery and can be performed at baseline, and 12 and 24 weeks of age. Following termination, eyes are processed for retinal histology and immunohistochemistry. Specifically, expression of RPGR (using a commercially-available antibody directed against human RPGR, but that does not recognize canine RPGR) can be used and its localization to the connecting cilium assessed. Photoreceptor integrity (including inner/outer segment structure, connecting cilium, and synaptic terminals) can be evaluated in RPGR expressing (i.e., treated) and non-expressing (i.e., untreated) areas and compared, using previously validated cell-specific antibodies. This can include using antibodies directed against (rhodopsin, and cone opsins) to examine correction of opsin mislocalization, but also of bipolar cells (e.g., PKCα and Goα) to assess the impact of photoreceptor rescue of inner retinal remodeling. Dosage information can be obtained, particularly dosages suitable for use with a 2 mm diameter area of the central XLPRA2/RPGR retina that confers photoreceptor rescue.

### Example 8

Dosing information gained from the example of above is used in this example. In particular, four dogs are used, and each animal has one eye implanted with a polymer-based gel implant (e.g., an OPO-AAV-RPGR gel implant), and the contralateral eye with a determined dose of a polymer-based gel implant (e.g., an OPO-AAV-GFP gel implant) that leads to detectable GFP expression in remaining photoreceptors during the early phase of the disease. Similar assessment methods as described above, including clinical ophthalmic exams, in vivo cSLO/OCT imaging, ERG, are performed every three months, and histology/assessment done at termination. In some embodiments, psychophysical visual training and testing of all dogs can be conducted using a well-established obstacle avoidance course and a forced 2-choice Y maze, which have both been successfully used to demonstrate rescued visual behavior after subretinal AAV-RPGR gene therapy in this model. These more advanced phases of degeneration in dogs can be used for modeling the situation encountered with human RPGR-XLRP patients who are frequently diagnosed when substantial ONL loss has already occurred. In addition, this example can evaluate the impact (positive or negative) of inner retinal remodeling on retinal permeability to AAVs released on the preretinal surface. In such embodiments, dogs are assessed every three months by eye examination, cSLO/OCT, ERG, visual behavior and histology/IHC at termination (90 weeks of age). If ONL rescue is seen with the 2 mm diameter polymer-based gel implant but there is minimal evidence of preserved visual function, then a wider retinal surface can be targeted using more of the polymer-based gel material. In some embodiments, subretinal delivery of a 150 μL volume covers approximately 60±13 mm² of the retinal surface in an adult (≥12 week-old) dog.

### Example 9

The RPE65 dog model of LCA2 can be used in this example to validate that the disclosed polymer-based gel implant embodiments can be used to target diseases that affect the RPE. As rod function is severely impaired from birth in these dogs due to lack of RPE65 isomerase activity in the RPE, restoration of RPE65 expression via gene augmentation can rapidly correct the visual deficit and be quantifiably assessed by ERG and visual behavior. In this example, an AAV that efficiently targets the RPE after intravitreal delivery can be used to package the human RPE65 cDNA (1602 nt) under control of the 823-nt human RPE65 promoter. A similar experimental design as described in Example 8 can be used, except that dogs are treated at 12 weeks of age, when the size of the globe is sufficiently large (axial globe length: 16-18 mm) to enable surgical implantation of large (25 mm²) polymer-based gel implants that can be tiled together to cover a retinal surface comparable to that targetable by subretinal injection of an AAV solution. Dogs are monitored by eye examination, cSLO/OCT imaging, and ERG for 18 weeks post-implantation. At termination, eyes are processed for histology to evaluate any potential deleterious effect of the retina, and to assess reduction of RPE inclusion and expression of RPE.

### Example 10

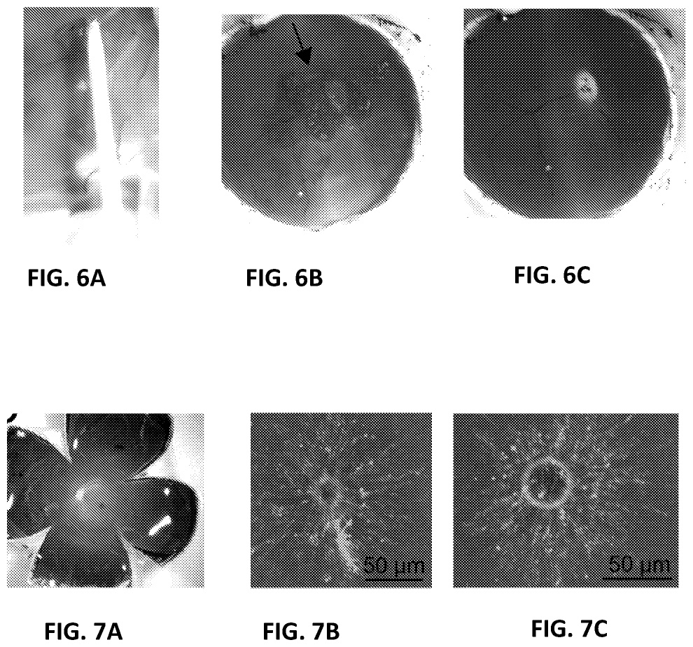

In this example, an OPO gel implant was tested ex vivo in pig eyes for retinal adhesiveness and for timing of release. OPO loaded with Fast Green dye for easier visualization was placed onto retinas of eyes kept at 37° C. using a soft-tipped cannula (FIG. 6A). One minute after the implant adhered to the retina, the eye was vigorously shaken to confirm adhesion (FIG. 6B). By 6 hours post placement the OPO gel implant had completely transitioned to a liquid phase, releasing the dye into the vitreous cavity (FIG. 6C).

### Example 11

In this example, 7m8-CAG-GFP is loaded into a 25% (w/v) OPO gel material and implanted in primate retinas. First, the material is tested ex vivo in primate eyes for retinal adhesiveness and for the timing of release. The gel implant is then placed onto maculae of eyes kept at 37° C. One minute after the implant adhered to the retina, the eye is vigorously shaken. The gel implant should remain in place. Fifteen minutes after placement, the implant is still in place above the macula and present as a gel. Next, implant procedures are performed in two primates. Two dosages are tested. The first experiment is a safety study for the gel implant material, and a minimal amount of AAV is loaded into the implant (5E+8 vector genomes). This low dosage is 0.5 log lower than the lowest dosage tested in Phase I/IIa XLRS clinical trials and 3 logs lower than dosages of 7m8 previously tested intravitreally in primates. The procedure is then performed as follows: 1) Limited conjunctival peritomy overlying the sclerotomy site; 2) Diathermy over the sclerotomy site for hemostasis; 3) Strategic transconjuctival placement of 3 working ports: infusion, chandelier, instrument; 4) Three port pars plana limited posterior preretinal vitrectomy; 5) Placement of gel implant above macula by injection. Two months after implantation, retinas are flat mounted and imaged. Dissection of the eye should reveal that the retinas are healthy, and no trace of the implant remains (FIG. 7A). Flat mount imaging of retinas should reveal that, even with this very low dose, GFP expression is apparent in the fovea of both eyes (FIGS. 7B and 7C) under the area where the gel implants are placed, but not in peripheral regions, indicating that vector is successfully encapsulated, released, and directed to the macula. A second primate can undergo a procedure to implant the OPO gel implant loaded with 100× larger dosage of AAV (5E+10 vg) and immune response monitored.

### Example 12

In this example, nine NPHP5-LCA mutant dogs are injected epiretinally at the time of disease onset (˜6 weeks of age) with an OPO gel implant loaded with AAV-NPHP5 viral particles at one of 3 concentrations (1×, 10×, and 100×) predetermined based on results conducted in WT dogs. Dogs are then randomly allocated to one of the 3 treatment groups (n=3 dogs/group). OPO-AAV is administered over a 50 mm² surface of the central retina, which is comparable to the surface of retina treated after a 150 μL subretinal injection in the dog. In all treatment groups, dogs will have their contralateral eye injected with the same type of OPO gel implant loaded with an AAV-TdTomato construct (same 3 doses as for AAV-NPHP5). This negative control allows one to verify that the AAV tropism towards canine PRs (established in WT dogs) is retained in diseased/mutant retinas. The 292-nt portion of the human GRK1 promoter, which has been shown to be effective driving transgene expression in both classes of PRs in dogs and in NHPs, is used. This pGRK1-NPHP5 payload is packaged in a lead AAV capsid variant to efficiently target rods and cones in NHPs, WT dogs, and human retinal explants. After epiretinal injection, the dogs are followed for 18 weeks. Weekly ophthalmic examinations including fundus photography is performed to monitor ocular tolerability to the gel implants. Longitudinal assessment of TdTomato expression (in the contralateral eyes) by fundus photography (custom-modified TopCon camera with set of filters to detect RFP), and structural integrity of the retinas by non-invasive cSLO/OCT imaging (Spectralis HRA/OCT2) is to be performed at pre-injection (˜6 wks of age=baseline), 12 weeks, and before termination at 24 weeks. In depth qualitative and quantitative analysis of ONL thickness is to be conducted after semi-manual segmentation of individual longitudinal reflectivity profiles from overlapping (30°×20°) raster OCT scans. Topographical maps of ONL thickness are examined to determine whether ONL rescue is seen in the area corresponding to the OPO-AAV injection. The dimension of the rescued region in relation to the injected volume of the OPO gel implant will inform on the extent of potential tangential diffusion of AAV out of the OPO gel implant and its ability to treat an area larger than where the gel implant is deposited. Full field ERG is used to assess any functional recovery of rod and cone function and will be performed at 6, 12 and 24 weeks of age. Multifocal ERG (mfERG) also can be performed, which enables visualization of the fundus via an integrated cSLO to circumvent the lack of fixation in an anesthetized animal. The use of mfERG is specifically indicated to detect focal cone dystrophy and has been used in the field to detect localized dysfunction in XLRP carriers that have patchy areas of degeneration as a result of random X-inactivation. Following termination, eyes are processed for in situ hybridization, retinal histology and immunohistochemistry. Expression of NPHP5 (using antibodies directed against human NPHP5 that do not recognize canine NPHP5)(83) is used and its localization to the connecting cilium is assessed. PR integrity (including inner/outer segment structure, connecting cilium, and synaptic terminals) is evaluated in NPHP5-expressing (treated) and non-expressing (untreated) areas and compared, using previously validated cell-specific antibodies. This includes using antibodies directed against rhodopsin and cone opsins to examine correction of opsin mislocalization, bipolar cells (e.g., PKCα and Goα) to assess the impact of PR rescue of inner retinal remodeling, and Iba-1, CD4, CD8 and CD20 antibodies to monitor any potential innate and adaptive cellular inflammation. Using this example, doses of epiretinally-delivered AAV—OPO-NPHP5 that confers PR rescue in the central retina can be determined.

### Example 13

In this example, a dose of OPO-AAV-NPHP5 as identified using Example 12 is selected and evaluated in NPHP5-LCA mutant dogs treated at ˜ 6 weeks of age and followed up to 90 weeks of age. Four dogs (2 males-2 females) are used, and each animal will have one eye injected pre-retinally with OPO-AAVNPHP5. The contralateral eye is injected with the dose of OPO-AAV-TdTomato that leads to detectable expression of this red fluorescent protein in remaining PRs during the early phase of the disease. Similar assessment methods as described above, including clinical ophthalmic exams, in vivo TopCon and cSLO/OCT imaging, ff and mfERG, are performed every 3 months, and histology/assessment done at termination. In addition, subcortical and cortical visual function is evaluated by respectively, pupillometry and fMRI, as well as functional vision using a well-established obstacle avoidance course and a forced 2-choice Y maze, which have both been successfully used in the art to demonstrate rescued visual behavior after subretinal AAV gene augmentation therapy in the NPHP5 and other canine models. This visual behavior text provides a highly sensitive method to demonstrate retention (or rescue) of central cones.

### Example 14

In this example, a similar protocol design as described above in Example 13 is implemented using 2 groups of 4 dogs treated at 14 weeks of age (mid-stage disease; ˜25% loss of PRs) and at 33 weeks of age (late-stage disease; ˜>50% loss of PRs). These more advanced phases of degeneration in dogs facilitate modeling the situation in human NPHP5-LCA patients who are frequently diagnosed when substantial ONL loss has already occurred, and islands of PRs persist only in the central retina. Further, the impact (positive or negative) of inner retinal remodeling on retinal permeability to AAVs delivered by epiretinal OPO injection is evaluated. Dogs are assessed every 3 months by cSLO/OCT, ffERG and mfERG, pupillometry, fMRI, visual behavior, and histology/IHC at termination (90 weeks of age). This protocol can be used to establish whether treatment of the canine central retina can rescue the remaining central PRs, cause central cones to regrow their outer segments, and restore both rod and cone-mediated functional vision.

### Example 15

In this example, RPE65-LCA2 dogs (age: 12 weeks) are used to validate that OPO-AAV can also target diseases affecting central RPE. As rod function is severely impaired from birth in these dogs due to lack of RPE65 isomerase activity in the RPE, restoration of RPE65 expression via gene augmentation can rapidly correct the visual deficit and be quantifiably assessed by ERG and visual behavior. Thus, it is expected that this model will provide a rapid readout of the success of OPO-AAV-RPE65 therapeutic intervention. An OPO-AAV embodiment for RPE identified in WT canine studies discussed herein is used to package the human RPE65 cDNA (1602 nt) under control of the 823-nt human RPE65 promoter. A similar protocol as described in Example 12 is followed (n=9 dogs; 3 dose groups, n=3 dogs/group) with the contralateral eye injected with OPO-AAV-TdTomato. Dogs are monitored by eye examination, Topcon fundus photography and cSLO/OCT imaging, and ffERG for 18 weeks post-injection. At termination, histology is performed to evaluate any potential deleterious effects/inflammation in the retina, and to assess reduction of RPE inclusions and expression of RPE65. As described in Example 12, the area of RPE corrected by treatment is estimated through serial sectioning.

### Example 16

In this example, dosing of OPO-AAV-RPE65 is evaluated to determine whether delivery of the OPO-AAV-RPE65 implant to the area centralis region of 4 RPE65 mutant dogs (2 males/2 females) at 2 years of age (before onset of central ONL loss) can stably restore rod and cone function and prevent PR degeneration over 78 weeks. Similar outcome measures as described in Example 14 are evaluated at baseline and every 3 months until termination.

In view of the many possible embodiments to which the principles of the present disclosure may be applied, it should be recognized that the illustrated embodiments are only examples and should not be taken as limiting scope. Rather, the scope of the present disclosure is defined by the following claims. We therefore claim as our invention all that comes within the scope and spirit of these claims.

1. A polymer-based gel implant, comprising:
a polymer component comprising one or more polymer species units, wherein the polymer component is capable of absorbing water such that the polymer component transitions from a gel phase to a liquid phase as a concentration of the polymer component in the polymer-based gel implant decreases; and
a therapeutic agent suspended in the polymer component; wherein the polymer-based gel implant is a gel at ambient temperature and comprises water.

2. The polymer-based gel implant of claim 1, wherein the polymer component has a structure satisfying a formula A-B-A, wherein B is a polymer species unit and each A independent is an end capping group attached to each end of the polymer species unit.

3. The polymer-based gel implant of claim 2, wherein each end capping group is an aliphatic group.

4. The polymer-based gel implant of claim 1, wherein the polymer species unit is a polyalkylene oxide.

5. The polymer-based gel implant of claim 1, wherein the polymer component is octadecane-poly(ethylene glycol)-octadecane and the polymer-based gel implant comprises more than 10% (w/v) water.

6. The polymer-based gel implant of claim 1, wherein the therapeutic agent is selected from a vector, a pharmaceutical drug, an optogenetic therapeutic agent, a naturally and/or non-naturally occurring CRISPR-Cas9 system, or any combination thereof.

7. The polymer-based gel implant of claim 6, wherein the vector is an AAV vector, a recombinant AAV vector, or any combination thereof.

8. The polymer-based gel implant of claim 6, wherein the vector is capable of infecting retinal cells, photoreceptor (rod and/or cone) cells, retinal ganglion cells, RPE cells, Müller cells, retinal pigmented epithelial cells, bipolar cells, amacrine cells, astrocytes, microglia, pericytes, vascular endothelium cells, horizontal cells, and other cells located in the ocular region.

9. The polymer-based gel implant of claim 1, wherein the therapeutic agent is suspended in the polymer component, which is in gel form.

10. The polymer-based gel implant of claim 1, further comprising a backing layer material that facilitates unidirectional delivery of the therapeutic agent from the polymer component such that the therapeutic agent does not pass through the backing layer material.

11. The polymer-based gel implant of claim 10, wherein the backing layer material comprises one or more polymer species units that are the same as the one or more polymer species units of the polymer component and wherein the one or more polymer species units of the backing layer have a different number of crosslinks as compared to the one or more polymer species units of the polymer component.

12. The polymer-based gel implant of claim 10, wherein the backing layer material comprises one or more polymer unit species that does not absorb water.

13. The polymer-based gel implant of claim 1, wherein the polymer-based gel implant becomes transparent upon exposure to an aqueous environment.

14. The polymer-based gel implant of claim 1, wherein the polymer component is octadecane-poly(ethylene glycol)-octadecane and the therapeutic agent is an AAV vector.

15. A method, comprising:
providing a polymer-based gel implant of claim 1; and
implanting the polymer-based gel implant into an ocular region of a subject.

16. The method of claim 15, wherein implanting is performed via injection.

17. The method of claim 16, wherein the injection is an intravitreal injection, a subretinal injection, or a combination thereof.

18. The method of claim 15, further comprising performing a partial or full vitrectomy.

19. The method of claim 15, wherein the method does not comprise removing the polymer-based gel implant or any degradation product formed therefrom.

20. A method, comprising treating a retinal disease and/or a retinopathy by implanting the polymer-based gel implant of claim 1 in an ocular region of a subject having, or capable of developing, the retinal disease and/or retinopathy.

21. The method of claim 20, wherein the retinal disease and/or a retinopathy is selected from central retinal vein occlusion, diabetic retinopathy, proliferative vitreoretinopathy, retinal arterial occlusive disease, retinal detachment, uveitic retinal disease, non-retinopathy diabetic retinal dysfunction, retinoschisis, retinitis pigmentosa, epiretinal membrane disorders, radiation retinopathy, retinal vein occlusion, chorioretinal degeneration, retinopathy of prematurity, acute macular neuroretinopathy, sympathetic opthalmia, Vogt Koyanagi-Harada syndrome, uveal diffusion, a posterior ocular condition, posterior ocular conditions, photocoagulation, branch anterior ischemic optic neuropathy, glaucoma, Usher syndrome, cone-rod dystrophy, Stargardt disease, inherited macular degeneration, Leber congenital amaurosis, congenital stationary night blindness, choroideremia, Bardet-Biedl syndrome, macular telangiectasia, Leber's hereditary optic neuropathy, and disorders of color vision, Behcet's disease, choroidal neovascularization, diabetic uveitis, histoplasmosis, macular degeneration, edema, multifocal choroiditis, ocular trauma, ocular tumors, and any combinations thereof.

22. The method of claim 20, wherein the retinal disease is RPE65-LCA2 or X-linked retinitis pigmentosa.

23. A method of making the polymer-based gel implant of claim 1, comprising:
hydrating the polymer component by combining it with water to provide a hydrated polymer component; and
combining the hydrated polymer component and the therapeutic agent to provide the polymer-based gel implant.
